(19) United States
(12) Patent Application Publication
(10) Pub. No.: US 2019/0365797 A1
(43) Pub. Date: Dec. 5, 2019

(54) Systems and Methods for Delivery of Nitric Oxide

(71) Applicant: ZYLO THERAPEUTICS, INC., GREENVILLE, SC (US)

(72) Inventor: Andrew Draganski, Greenville, SC (US)

(21) Appl. No.: 16/431,905

(22) Filed: Jun. 5, 2019

Related U.S. Application Data
(60) Provisional application No. 62/680,928, filed on Jun. 5, 2018.

(51) Int. Cl. A61K 33/00; A61K 9/00; A61K 9/06; A61K 47/06; A61K 47/46

(52) U.S. Cl. CPC A61K 33/00; A61K 9/0014; A61J 1/00; A61K 47/06; A61K 47/46; A61K 9/06

(57) ABSTRACT

The present disclosure is directed to systems and methods for delivery of nitric oxide (NO). The methods and systems disclosed herein may provide advantages by controlling NO generation until desired by an end user. To achieve controlled NO generation, embodiments of the disclosure can include multiple compositions that may be in the form of a kit or a multi-chamber delivery system. Additionally, the disclosure contemplates the use of these systems as methods for generating NO by mixing the multiple compositions to yield the controlled generation and/or release of NO. Methods of the disclosure can be used for the controlled delivery of NO to a biological surface (e.g., the skin) and may be used to treat a condition or disease.

### CROSS REFERENCE TO RELATED APPLICATION

This application claims filing benefit of U.S. Provisional Application Ser. No. 62/680,928, having a filing date of Jun. 5, 2018, which is incorporated herein by reference in its entirety.

### FIELD OF THE INVENTION

The present disclosure is directed to delivery systems which include stabilized nitric oxide-generating compositions (e.g., two-part delivery systems), methods for generating or delivering nitric oxide (NO), and synthesis methods employed for preparing the compositions and the nitric oxide-donating sources.

### BACKGROUND

Research focusing on nitric oxide therapy has demonstrated that nitric oxide has the potential for use in treatment of many medical conditions, including: inflammation, cardiac ischemia, erectile dysfunction, atopic dermatitis, and even certain forms of cancer. However, a significant challenge to developing viable nitric oxide therapies is overcoming the chemical instability of NO-donor sources (e.g., S-nitrosothiols and nonoate functional groups). Two of the more common chemistries utilized as NO-donors are S-nitrosothiols and nonoates, both of which are susceptible to releasing NO prematurely upon exposure to mild conditions. For example, in the case of S-nitrosothiols, ambient heat, ambient light, or exposure to moisture can be enough to release NO prematurely.

Accordingly, one of the objects of the present disclosure is to provide shelf-stable NO-donor formulations which can provide sustained release of NO with topical application.

### SUMMARY OF THE INVENTION

The present disclosure is directed to systems and methods for delivery of nitric oxide (NO). The methods and systems disclosed herein may provide advantages by controlling NO generation until desired by an end user. To achieve controlled NO generation, embodiments of the disclosure can include multiple compositions that may be in the form of a kit or a multi-chamber delivery system. Additionally, the disclosure contemplates methods for generating NO that include mixing the multiple compositions to yield the controlled generation and/or release of NO. Methods of the disclosure can be used for the controlled delivery of NO to a biological surface (e.g., the skin) and may be used to treat a condition or disease.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures.

FIG. 1 illustrates the particle size distribution of thiol-functionalized (blank) silica particles prepared in accordance with the disclosure.

FIG. 2 illustrates the particle size distribution for thiol-functionalized (blank) silica particles prepared in accordance with the disclosure. Prior to analysis, these particles were ground to smaller size with a mortar and pestle.

FIG. 3 illustrates the particle size distribution for thiol-functionalized (blank) silica particles prepared in accordance with the disclosure. Prior to analysis, the particles were ground to smaller size with a mortar and pestle.

FIG. 4 illustrates the particle size distribution for thiol-functionalized (blank) silica particles prepared in accordance with the disclosure. Sample was ground using a mortar and pestle; sample was sonicated during loading and during analysis.

FIG. 5 illustrates the particle size distribution for thiol-functionalized (blank) silica particles prepared in accordance with the disclosure. Sample was ball-milled using 1 mm ZrO₂ beads; sonication was performed at level 4 during loading and during analysis.

FIG. 6 illustrates the particle size distribution for thiol-functionalized (blank) silica particles prepared in accordance with the disclosure. Sample was ball-milled using 1 mm ZrO₂ beads; sonication was performed at level 8 during loading and during analysis.

FIGS. 7A and 7B illustrate graphs displaying NO release rate and cumulative NO release, respectively, versus time in accordance with an example embodiment of the disclosure.

FIG. 8 illustrates a graph displaying cumulative NO release in accordance with an example embodiment of the disclosure.

FIG. 9 displays a photograph of a two-compartment frangible pouch before and after mixing.

FIG. 10 displays a photograph of a two-chamber airless dip tube dispenser system.

FIG. 11 displays a photograph of a two-barrel syringe with mixing nozzle.

FIG. 12 illustrates the particle size distribution for thiol-functionalized (blank) silica particles prepared in accordance with the disclosure.

Repeat use of reference characters in the present specification and drawings is intended to represent the same or analogous features or elements of the present invention.

### DETAILED DESCRIPTION

Reference now will be made to the embodiments of the disclosure, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only and is not intended as limiting the broader aspects of the present invention, which broader aspects are embodied exemplary constructions.

The present disclosure is directed to systems and methods for delivery of nitric oxide (NO). The methods and systems disclosed herein may provide advantages by controlling NO generation until desired by an end user. To achieve controlled NO generation, embodiments of the disclosure can include multiple compositions that may be in the form of a kit or a multi-chamber delivery system. Additionally, the disclosure contemplates methods for generating NO that include mixing the multiple compositions to yield the controlled generation and/or release of NO.

As an example embodiment, a method for generating NO can include obtaining at least two compositions (e.g., a first composition and a second composition). One of the at least two compositions can include a thiol-bearing species. Another of the at least two compositions can include one or more compounds such as a nitrite. By mixing a portion of each composition together, the nitrite can react to form an S-nitrosated functionality upon mixing with the thiol-bearing species. Since S-nitrosated compounds can be unstable even under mild conditions, methods and systems of the disclosure can be used for applications or treatments without the need for high-pressure or cold storage. Thus, embodiments of the disclosure may provide benefits for consumers by reducing delivery and/or storage costs. Additionally, certain embodiments may be used in areas that do not have access to advanced storage systems.

One aspect of obtaining the at least two compositions can include preparing one or more of the at least two compositions. For example, obtaining a composition including a thiol-bearing species can include purchasing a solution or mixture containing a thiol-bearing species, preparing a solution or mixture containing a thiol-bearing species, or synthesizing a thiol-bearing species, or a combination thereof.

An aspect of the at least two compositions can include a carrier. Generally, the carrier can be in a form (e.g., a solution, a mixture, a liquid, an emulsion, a hydrogel, etc.) that is used to facilitate application and/or mixing of the at least two compositions. In certain embodiments, the carrier can be present in one or more of the at least two compositions. If the carrier is present in more than one of the at least two compositions, the carrier in each composition can be selected independently so that some, all, or none of the compositions may include the same carrier. Several non-limiting examples of carriers include petrolatum and aloe vera, though any biologically compatible and/or low-toxicity preparation may be used.

For certain embodiments, the thiol-bearing species can include a plurality of particles that each have one or more thiol groups (—SH) at the particle surface. The plurality of particles having thiol groups can be formed using the techniques described herein or may be formed via other synthesis techniques. Aspects of the plurality of particles can include size characteristics, such as particle diameter and/or population characteristics such as polydispersity. In some embodiments, the plurality of particles can be polydisperse. As used herein, polydisperse particles are characterized as having a standard deviation (S.D.) greater than or equal to about 10% of the mean particle size.

Alternatively, or additionally, the thiol-bearing species can include a polymer and/or a compound that includes a thiol group. Several non-limiting examples of a polymer or compound having a thiol group include: cysteine, peptides that include cysteine, N-acetyl cysteine, erythro- and threo-3-mercapto-2-methylbutanol, 2-mercapto-2-methylpentan-1-ol, 3-mercapto-2-methylpentan-1-ol, and ethyl 3-mercaptobutyrate.

Methods for generating NO in accordance with this disclosure can be accomplished using various arrangements. As an example, for some methods, the first composition including the thiol-bearing species can also include the nitrite and an acid in a substantially anhydrous form. In other methods, another of the two or more compositions (e.g., a second composition) can include the nitrite. To facilitate nitrosation, in certain methods an acid and/or water can be included in at least one of the two or more compositions.

Example arrangements in accordance with the disclosure can include: (1) a first composition containing the thiol-bearing species and the nitrite, and a second composition containing an acid; (2) a first composition containing the thiol-bearing species, and a second composition containing an acid; (3) a first composition containing the thiol-bearing species and an acid, and a second composition containing the nitrate; and (4) a first composition containing the thiol-bearing species, a nitrite, and an acid, in addition to a second composition including water.

In some embodiments, a water scavenger or a desiccation agent may be used to maintain lower water concentrations in one or more of the compositions. For example, to achieve a substantially anhydrous composition in certain embodiments, a desiccant such as silica may be included in one of the two or more compositions or within a compartment containing the composition.

Another embodiment of the disclosure includes a multi-chamber delivery system. The multi-chamber delivery system can include a first chamber that contains a polydisperse mixture of thiol-bearing particles, a second chamber that contains one or more compounds, and a barrier designed to physically prevent material contained in the first chamber from contacting material contained in the second chamber until a condition is satisfied.

In some embodiments, the first chamber may contain the polydisperse mixture of thiol-bearing particles as a composition. Example compositions that may be contained in the first chamber can include any of the compositions that are disclosed herein, which include a thiol-bearing bearing species. For instance, in addition to the polydisperse mixture of thiol-bearing particles, the first chamber can include one or more of a carrier, a nitrite, or an acid.

Aspects of the barrier can include durability. In some implementations, a breakable barrier (e.g., a frangible seal) may be used to allow material in the first chamber to contact material in the second chamber to generate NO. In other implementation, the barrier may be durable, and the inclusion of a mixing region may be necessary to allow material from the first chamber to contact material from the second chamber. Thus, the condition can include shearing a seal to breach the barrier or using a pump to force material from the first chamber and material from the second chamber to move to a mixing region.

To facilitate pumping and/or mixing of material in the first chamber with material in the second chamber, a carrier can be included in accordance with embodiments of the disclosure.

Another embodiment of the disclosure can include a method for providing extended-release delivery of nitric oxide to a biological surface. NO has several biological effects, and the delivery systems and methods of this disclosure can also be applied to provide extended release delivery of NO to a biological surface (e.g., the skin) for use as a treatment or as a form of topical delivery.

A biological surface is not limited to solely include the skin and this disclosure contemplates the NO delivery to mucous membranes, keratin surfaces (e.g., nails, hair etc.), and vasculature (e.g., by injection.)

An example aspect of applying the mixture, the first composition, or the second composition to the biological surface can include treating a condition. Thus, for certain embodiments, methods for delivering NO to a biological surface can be used to treat or ameliorate a condition such as one or more of the following: inflammation, cardiac ischemia, erectile dysfunction, atopic dermatitis, and cancer.

In general, the conditions described herein are meant to be exemplary and are not intended to limit additional conditions that delivery of nitric oxide may be used to treat. Several additional conditions that can be treated by delivery of nitric oxide include: fungal infections of the skin and nails such as onychomycosis, general wound healing, treatment of and prevention of infection or infected wounds especially those caused by methicillin resistant Staphylococcus aureus (MRSA), acne vulgaris, and bacterial infections of animals including rain rot in horses. Therefore, for some applications, the biological surface need not be limited to only humans and may be extended to include portions of animals such as coats, hides, hooves, or other areas.

One aspect of the disclosure relates to compositions comprising silicate microparticles and/or nanoparticles (herein referred to as “silicate particles”) that can provide controlled and extended release of nitric oxide. It should be noted that the particles generated by certain methods of this disclosure are not monodisperse (i.e., polydisperse, and they cannot be characterized as being of approximately the same or similar particle size/diameter). For example, it cannot be said that these particles are approximately 200, or 400, or 800 microns in diameter. Instead, certain implementations of the present disclosure may include or otherwise utilize particles having a distribution of particle sizes.

Methods for providing the extended or controlled release of nitric oxide can include characterization of a release rate characterizing the amount of NO delivered over time. Additionally, or alternatively, the cumulative release of NO can be used to characterize the amount of NO released (e.g., expressed as a percentage based on the total amount of NO or nitrite) over a period of time. For example, certain embodiments of the disclosure can include methods for the extended release delivery of NO. In some of these embodiments, the cumulative release of NO can demonstrate delivery of about 50% of the NO within about 10 hours. In certain embodiments, the cumulative release of NO can demonstrate delivery of about 75% of the NO within about 24 hours. Since the NO release rate can be modified by adjusting the delivery conditions (e.g., by delivering the composition as an aqueous solution or mixture), in some embodiments the cumulative release of NO can demonstrate delivery of about 50% of the NO within about 3 hours.

Another aspect of the disclosure can be the ratio of certain components included in the example delivery systems. For example, the amounts of the nitrite compound and the thiol-bearing species may be adjusted to modify the ratio of thiol groups included in the thiol-bearing species to the NO produced by the nitrite compound. Without intending to be bound to one theory, the equilibrium of free NO and S-nitrosothiol groups can be used to adjust the NO release rate. As an example, for embodiments having a less than 1:1 ratio (e.g., 1:2) of thiol-bearing groups to NO produced by the nitrite compound, the amount of NO generated can exceed the number of thiol-bearing groups which may increase the NO delivery rate (e.g., less NO exists in the bound S-nitrosothiol form). As another example, for embodiments having more than a 1:1 ratio (e.g., 2:1) of thiol-bearing groups to NO produced by the nitrite compound, the amount of NO generated can fall short the number of thiol-bearing groups which may decrease the NO delivery rate (e.g., more NO exists in the bound S-nitrosothiol form). Thus, for embodiments of the disclosure, a range of ratios of the thiol-bearing compound to the nitrite compound may be used depending on the application and delivery profile. Ranges of the ratio of thiol-bearing groups to NO produced by the nitrite compound can include about 1:5 to about 8:1, such as about 1:4 to about 6:1, about 1:2 to about 4:1, and about 1:1 to about 2:1.

As used herein, “acid” is a proto donor that participates in the nitrosation reaction. An organic acid generally refers to an organic compound or higher molecular weight species such as a polymer, which exhibits a labile acid group (e.g., a carboxylic acid). An acid may also refer to an inorganic acid such as hydrochloric acid or phosphoric acid. The acid may be selected from the group consisting of alpha hydroxy acids (AHA's), amino acids, acid-terminated oligomers, acid-terminated dendrimers (comprising 3 or more acid terminal functional groups), acid-terminated linear and branched polymers, inorganic acids (e.g., HCl), and combinations thereof. Suitable AHA's may include citric acid, lactic acid, glycolic acid, malic acid, and AHAs that are accepted—and considered safe—for use in the cosmetic industry. Other suitable acids include benzoic acid and salicylic acid. Acids which are not suitable for use include those that are harsh and cause skin irritation, or acids such as ascorbic acid which are not acidic enough to fully nitrosate the available thiol groups in therapeutic formulations.

In certain implementations, an acid-terminated PEG functions as the acid for nitrosation. This acid is optionally used in combination with one or more supplementary acids. The PEG may comprise one or a plurality of carboxylic acid terminal groups (monofunctional or difunctional, respectively), depending on the type of PEG used: A four arm PEG, for example, may exhibit 4 terminal acid groups, and an 8 arm PEG may exhibit 8 terminal acid groups. Due to the hydrophilic nature of polyethene glycols, an acid-terminated PEG may be incorporated into an aqueous solution or gel. In biphasic emulsion systems, the acid-terminated PEG may be incorporated into the hydrophilic phase of a water-based cream. For example, a biphasic cream comprised of 70% water and 30% hydrophobic component can be mixed with the acid-terminated PEG, which will readily partition to the water phase of the cream.

As used herein, a carrier can refer to the disperse phase of that may be included in compositions disclosed herein, such as part A or part B in the example two-part delivery systems described below. Suitable examples of carriers include but are not limited to, petrolatum-based ointments, water-based (aqueous) hydrogels, siloxane and silicone gels, multiphasic vehicles (comprising both hydrophobic and hydrophilic phases in a single ointment, or cream, for example). It is important to note that parts (A) and (B) of two-part systems can comprise different delivery vehicles and will form a finished mixed product, e.g., (A) could be an anhydrous siloxane or ointment, while (B) is an aqueous hydrogel.

### Example Two-Part Systems

An example aspect of the disclosure is directed to multi-part systems (e.g., a two-part system having parts A and B) for delivering therapeutic compositions comprising activated nitric oxide-donors. The two-part design can provide stability through physical separation of “blank” thiol-bearing species (particles, compounds, polymeric materials, etc.) from nitrite source, acid, and/or water. The physical separation can be used to prevent premature nitrosation, which in certain preparations does not occur until parts A and B are mixed just before application.

The two-part system may utilize a double syringe (with or without a mixing tip), separate resealable tubes, dual dispensing pump action container comprising two separate reservoirs, etc. The two-part system may utilize a two-compartment pouch having a frangible seal that upon application of pressure breaks so that the two parts may be mixed together.

The two-part systems may comprise various combinations of chemical components. The two parts are referred to herein as parts A and B. For example, various arrangements of two-part systems are disclosed below:
1. A two-part system comprising a first part A and a second part B, wherein part A comprises blank particles, a nitrite salt, and an ointment.
2. A two-part system comprising a first part A and a second part B, wherein part B further comprises an acid dispersed within a vehicle, wherein said vehicle may be an ointment, a cream, a biphasic cream, an aqueous solution, a hydrogel, aloe vera gel.
3. A two-part system comprising a first part A and a second part B, wherein the A comprises blank particles and an acid, and wherein part B comprises a nitrite salt.
4. A two-part system comprising a first part A and a second part B, wherein part A comprises blank particles, a nitrite source (e.g., nitrite salt and/or alkyl nitrite), and an acid, and wherein part B comprises water and part A is an anhydrous mixture.
5. Any of the above two-part systems with the addition of a third part C, wherein part C is composed of an alkaline material such that when mixed with the previously mixed parts A+B, has the effect of neutralizing the residual acid to bring the final mixture to a preferred pH.

In one aspect of the disclosure, the two-part delivery system includes two separate carrier vehicles (a few examples of carrier vehicles are ointments, creams, emulsions, multiphasic topical creams), wherein the two separate carrier vehicles primary function is to physically separate the thiol-bearing species (e.g., silicate particles) from the activating acid/nitrite combination. This separation of chemicals can be accomplished in multiple ways: The thiol species can be mixed into carrier vehicle “A” in combination with the nitrite source and/or the acid source. Note that it is possible to combine the thiol species, nitrite source and acid into part “A”; however, it is imperative that the formulation is anhydrous so that premature nitrosation cannot occur. The second carrier vehicle, part “B,” can comprise the nitrite source and/or the acid source. Optionally, part “B” contains neither the nitrite source nor the acid, and only provides water in order to activate the nitrosation reaction upon mixing with part “A.”

In another aspect, part “A” is petrolatum-based hydrophobic ointment comprising thiol-bearing silicate particles and sodium nitrite, wherein nitrite and thiol groups are present in equimolar amounts. According to the same embodiment, part “B” is an aqueous-based hydrogel comprising an equimolar amount of acid relative to both thiol and nitrite. Preferably, the nitrite is sodium nitrite, and the acid is an alpha hydroxy acid that is hydrophilic and relatively soluble in the aqueous environment of part “B.” In this preferred embodiment, the thiol-bearing silicate particles do not become nitrosated until parts “A” and “B” are mixed (ideally, just prior to application or treatment), at which point the mixture of both parts turns red to indicate activation (formation of S-nitrosothiol groups on the silicate particles). The color indicator serves to reassure the user that the composition is still active and will release the therapeutic dose of nitric oxide.

In another aspect, part “A” is petrolatum-based hydrophobic ointment comprising thiol-bearing silicate particles and anhydrous acid, wherein the acid and thiol groups are present in equimolar amounts. According to the same embodiment, part “B” is an aqueous-based hydrogel comprising an equimolar amount of nitrite relative to both thiol and acid. Nitrite may be in the form of an inorganic salt, such as sodium nitrite, or in the form of an alkyl nitrite, such as tert-butyl nitrite. The acid may be an alpha hydroxy acid, such as citric acid, but other acids detailed in the present disclosure may be suitable. Ideally, the acid is soluble in the hydrophobic environment of part “A.” In this preferred embodiment, the thiol-bearing silicate particles do not become nitrosated until parts “A” and “B” are mixed (ideally, just prior to application or treatment), at which point the mixture of both parts turns red to indicate activation (formation of S-nitrosothiol groups on the silicate particles). The color indicator serves to reassure the user that the composition is still active and will release the therapeutic dose of nitric oxide.

In another aspect, the thiol source, anhydrous acid and nitrite source are mixed together in the hydrophobic gel, and the aqueous gel serves as the activator. It is imperative that the hydrophobic gel remain anhydrous during storage to prevent interaction between acid and nitrite so that RSNO does not form prematurely. Preferably the hydrophobic gel is petrolatum based. Preferably the acid is citric acid or other alpha hydroxy acid, an amino acid or a combination thereof. The nitrite may be a nitrite salt such as sodium nitrite, or an alkyl-nitrite where alkyl may be a straight chain or branched alkyl group, such as tert-butyl nitrite.

In another aspect, an anhydrous hydrophobic ointment is loaded with thiol functionalized silicate particles, sodium nitrite, and citric acid (each present in equimolar amounts). In this composition there is minimal nitrosation due to an absence of water an inability of citric acid to effectively function as an acid. The composition can be activated by addition of water or by mixture with a water source provided by part “B” (e.g., an aqueous gel such as aloe vera gel, or simply water alone).

In another aspect of the disclosure, nitrosated silicate particles and an acid are dispersed within a two-phase cream or ointment delivery vehicle. The formulation comprising the vehicle, particles and acid exists such that the particles are localized to the hydrophobic phase of the vehicle and the acid is localized to the hydrophilic aqueous phase, thereby keeping the particles and acid phase separated. This ensures minimal interaction between acid and nitrosated particles—which would result in premature nitric oxide release—while allowing a cream or ointment to be packaged in a single container or tube. When the cream or ointment is applied topically, the two phases are physically mixed on the skin surface and particles are nitrosated and capable of delivering nitric oxide.

In another aspect, blank thiolated organosilicate particles can be prepared as described in Example 2 and Example 4. The blank particles are incorporated into a delivery vehicle such as an ointment, wherein the particles account for a minor fraction of the total formulation (i.e., less than 50% by weight), and preferably wherein the particles account for approximately 20% w/w. An equimolar amount of nitrite (in the form of sodium nitrite), relative to the moles of thiol groups contained in the organosilica particles, is incorporated into the ointment. The sodium nitrite can be dissolved in water or added as an anhydrous salt and mixed in using a mortar and pestle. An equimolar amount of acid, in the form of citric acid, is thoroughly mixed into the ointment as well. In one preferred embodiment, the acid is anhydrous. Water (about 5% by weight of the total mixture) is added to activate the formulation and cause nitrosation of thiol groups while also changing the color from white to dark pink/red. In another embodiment, the acid is added as an aqueous solution to activate part “A,” which initially is devoid of acid.

Different variations of the two-part system were prepared and tested for nitric oxide release (indicated by “efficiency”). The results are tabulated below:

| WD2 | TM/Oint/1x cit | TE/Oint/1x Asc. | TE/Oint/1x Cit. | TE/Oint/HCl | TE/Cream/1x Cit. |
|---|---|---|---|---|---|
| Precursor | TMOS | TEOS | TEOS | TEOS | TEOS |
| Vehicle | Ointment | Ointment | Ointment | Ointment | Biphasic Cream |
| Acid | Citric (1.1x) | Ascorbic (1.1x) | Citric (1.1x) | HCl (1.1x) | Citric (1.1x) |
| (molar eq.) | 72.10% | 8.34% | 74.90% | 63.70% | 35.60% |
| Efficiency | | | | | |
| WD2 | TE/Oint/.8x | TE + HCl | TE + Ascorbic | TE/.33 citric/ | TE/.67x citric/oint |
| Precursor | NO2/Cit | TEOS | TEOS | oint | TEOS |
| Vehicle | TEOS | Ointment | Ointment | TEOS | Ointment |
| Acid | Ointment | HCl (1.1x) | Ascorbic (1.1x) | Ointment | Citric (0.67x) |
| (molar eq.) | Citric (0.8x) | 88.60% | 24.30% | Citric (0.33x) | 52.3% |
| Efficiency | 49.10% | | | 40.2% | |
| WD2 | 1x citric/oint | TM/.67 citric/ | TE/Aloe/Citric | | |
| Precursor | TEOS | cream | TEOS | | |
| Vehicle | Ointment | TMOS | Aloe Vera | | |
| Acid | Citric (1x) | Biphasic | Citric (1.1x) | | |
| (molar eq.) | 74.7% | Cream | 82.90% | | |
| Efficiency | | Citric (0.67x) | | | |
| | | 50.70% | | | |

Another aspect of the disclosure relates to thiol-bearing chemical species that may serve as alternatives or can be included in addition to the aforementioned silicate particles. In one embodiment, cysteine or a cysteine analog, such as N-acetyl cysteine, is incorporated into compositions as the thiol-bearing species. In another aspect, cysteine or cysteine analogs compliment the silicate particles and serve as a second source of thiol. The activation strategy for cysteine-based compositions is analogous to that employed for the silicate particles: The cysteine is housed in one compartment of the two-part delivery system, and the activating chemical or chemicals is housed in the second compartment to keep the cysteine physically separate from the activator. Nitrosation of cysteine does not occur until the time of application when the two components are mixed.

In some embodiments, the thiol-bearing chemical species that may be included with or used as alternative to the aforementioned silicate particles may be a secondary or tertiary thiol. As an example embodiment, penicillamine can be incorporated into compositions as the thiol-bearing species. In another example embodiment, penicillamine compliments the silicate particles and serves as a second source of thiol. Activation for penicillamine-based compositions is generally analogous to that employed for the silicate particles: The penicillamine can be housed in one compartment of a multi-part (e.g., two-part) delivery system, and the activating chemical or chemicals can be housed in the second compartment to keep the penicillamine physically separate from the activator. Nitrosation of penicillamine does not occur until the time of application when the two components are mixed.

It is recognized that in certain forms of therapy, bioactive agents/drugs may be added to formulation to provide an enhanced therapeutic effect, or a synergistic effect, in combination with nitric oxide.

The following examples discusses various methods and procedures and provides exemplary embodiments that may be understood in conjunction with the Drawings and Description provided herein. The specific methods and procedures described in the examples are not meant to limit the disclosure and are provided solely to illustrate some of the ways in which the invention may be practiced

### Examples

### Example 1. Preparation of S-Nitrosothiol-Functionalized Silicate Particles, from TMOS and MPTS

Co-condensed silicate particles were prepared from mercaptopropyl trimethoxylsilane (MPTS) and tetramethoxysilane (TMOS). The hydrolysis reaction was carried out in separate vials for the two monomers: 600 ul MPTS was hydrolyzed at room temperature for 1.5 hours in a 15 ml plastic centrifuge tube in the presence of methanol (4.8 ml), hydrochloric acid (24.6 ul of 100 mM HCl), and water {1.1754 ml). TMOS was hydrolyzed in a separate vial using 1.5 ml of TMOS and 330 ul of 10 mM HCl. Prior to the addition of acid, the TMOS was chilled on ice for 10 minutes. 330 ul of 10 mM HCl was subsequently added to the chilled TMOS, which immediately separated into two phases. The mixture was vortexed vigorously then sonicated for approximately 120 seconds, at which point one phase was formed. The formation of a single phase indicating sufficient hydrolysis of the monomer (the hydrolysis reaction generated methanol which increased solubility of silanes in the reaction medium). The vial containing hydrolyzed TMOS was placed back on ice. Next, the hydrolyzed MPTS was nitrosated by dissolving 250 mg sodium nitrite into the MPTS solution, followed by the addition of 267 ul of 12 mM HCl to create S-nitrosothiol (RSNO) functional groups. The MPTS solution turned dark red upon formation of SNO and was vortexed vigorously. Quickly, but carefully, 750 ul of PEG 400 was combined with 12 ml of phosphate buffer (pH 7.4), added to the MPTS solution and vortexed. Then the hydrolyzed TMOS solution was combined with the MPTS solution, quickly vortexed, and within 10 minutes, a spanning network gel was formed. The sample was stored in the freezer overnight prior to the lyophilization step.

### Example 2. Preparation of “Blank” Thiol-Functionalized Silicate Particles, from TMOS and MPTS

Co-condensed silicate particles were prepared from mercaptopropyl trimethoxylsilane (MPTS) and tetramethoxysilane (TMOS). The hydrolysis reaction was carried out in separate vials for the two monomers: 600 ul MPTS was hydrolyzed at room temperature for 1.5 hours in a 15 ml plastic centrifuge tube in the presence of methanol (4.8 ml), hydrochloric acid (24.6 ul of 100 mM HCl), and water {1.1754 ml). TMOS was hydrolyzed in a separate vial using 1.5 ml of TMOS and 330 ul of 10 mM HCl. Prior to the addition of acid, the TMOS was chilled on ice for 10 minutes. 330 ul of 10 mM HCl was subsequently added to the chilled TMOS, which immediately separated into two phases. The mixture was vortexed vigorously then sonicated for approximately 120 seconds, at which point one phase was formed. The formation of a single phase indicating sufficient hydrolysis of the monomer (the hydrolysis reaction generated methanol which increased solubility of silanes in the reaction medium). The vial containing hydrolyzed TMOS was placed back on ice. Next, 750 ul of PEG 400 was combined with 12 ml of phosphate buffer (pH 7.4), added to the MPTS solution and vortexed. Then, the hydrolyzed TMOS solution was combined with the MPTS solution, quickly vortexed, and within 10 minutes, a spanning network gel was formed that appeared white and opaque. The sample was stored in the freezer overnight prior to the lyophilization step.

### Example 3. Preparation of S-Nitrosothiol-Functionalized Silicate Particles, from TEOS and MPTS

A sol-gel was prepared according to a similar procedure as disclosed in Example 1. However, instead of TMOS, TEOS was used as the co monomer with MPTS, and different hydrolysis conditions were required for TEOS:

MPTS was hydrolyzed as described in Example 1.

2.25 ml TEOS was hydrolyzed in the presence of 3.45 ml of ethanol and 2.00 ml of 10 mM HCl, which was mixed followed by extensive vortexing. TEOS hydrolyzed in less than 10 minutes. The rest of the procedure was followed generally according to what is disclosed in Example 1, with the following adjustments: There was also an adjustment to the final mixture: 0.75 ml PEG 40, 6 ml 0.1 M phosphate buffer at pH 7.4, and 1 ml of 0.1 M NaOH. Because of the extra ethanol needed in the TEOS hydrolysis step, the amount of buffer was reduced in the final mixture so that the final volume would be consistent. Also, because of the additional acid in the TEOS hydrolysis step, an aliquot of NaOH base was added to counteract the additional acid. pH was measured in the final mixture and was found to be around 7.4. The final mixture was held for 60 minutes to allow the spanning network to form.

### Example 4. Preparation of “Blank” Thiol-Functionalized Silicate Particles from TEOS and MPTS

Particles were prepared as described in Example 3, with the exception being that nitrosation of MPTS was not performed. This resulted in a white, opaque gel, which was stored in the freezer overnight prior to lyophilization. This “blank” gel formed slightly slower than the gel disclosed in Example 3.

### Example 5. Preparation of “Blank” Thiol-Functionalized Silicate Particles from TEOS and MPTS

A silica sol-gel monolith containing mercaptopropyl functional groups was synthesized by performing independent hydrolysis of silica monomers tetraethoxysilane (TEOS) and 3-mercaptoproyltrimethoxysilane (MPTS), followed by monomer condensation to form a spanning network. MPTS hydrolysis was initiated by combining the following: 3.2 milliliters (ml) Methanol, 0.655 ml deionized (DI) H₂O, 0.153 ml 0.1N hydrochloric acid (HCl), and 0.4 ml MPTS, and vortexing to mix well. The MPTS hydrolysis reaction was allowed to proceed at room temperature (RT) undisturbed for 90 minutes. TEOS hydrolysis was initiated 30 minutes after starting MPTS hydrolysis by combining the following in a separate vessel: 1.24 ml anhydrous Ethanol, 0.655 ml DI H₂O, 0.073 ml 0.1N HCl, 0.8 mL TEOS, and vortexing to mix well. The TEOS hydrolysis reaction was allowed to proceed undisturbed for 60 minutes at room temperature. At the end of hydrolysis, the MPTS and TEOS hydrolysates were combined and vortexed to mix. Then, condensation (sol-gel monolithic spanning network formation) was initiated by adding 5.8 ml of sodium phosphate buffer (0.1 M, pH 7.4) and vortexing to mix well. The mixture was held at room temperature for a 4-hour period for condensation. Following condensation, the sol-gel was dispersed into discrete silica particles by application to a vortex mixer on high setting. In this example, the mole % of MPTS was 37.4% (balance TEOS).

### Example 6. Lyophilization of Sol-Gels

The spanning network gel (as formed in examples 1 and 2), was placed in the −80° C. freezer for approximately 30 minutes before lyophilization. When 30 minutes elapsed, the sample was quickly transferred to a glass sublimation chamber, attached to the running lyophilizer, and pressure was immediately reduced by the connected vacuum pump. The pressure stabilized at approximately 0.300 mbar which was sufficient to pull off the residual solvent in about 6 hours. At the end of lyophilization the sample had formed a dry powder. A mortar and pestle were used to grind the powder into fine particles, and then the sample was stored in a freezer at −20° C. until further use.

### Example 7. Nitrosation of “Blank” Thiol-Bearing Particles

Following lyophilization, “blank” particles were mixed into petrolatum-based ointment at 20% w/w. To the homogenous paste, an equimolar amount of sodium nitrite was added and blended thoroughly. An equimolar amount of acid (calculated based on moles of acidic protons per mole of acid) was then added and mixed into the ointment to nitrosate the thiol-bearing particles. Within 10 seconds of adding and mixing the acid into ointment, the entire mixture changed color from white/off-white to dark pink/red.

### Example 8. Nitric Oxide Release

The nitric oxide release was quantified immediately following nitrosation as described in Example 4. Approximately 50-150 mg of the activated composition (the ointment blended with nitrosated particles) was weighed and added to a pear-shaped 50 ml flask that was connected in-line with the nitric oxide detector for a chemiluminescence detector, CLD 60. The flask was designed as the sample chamber and included a port for nitrogen gas, a port for sample addition, and a vacuum port connected directly to the detector. For ointment-based samples, 3 ml of light mineral oil was added to the flask to facilitate nitric oxide release from the sample. Mineral oil was added to the flask first, allowed to purge with nitrogen as the CLD 60 pulled low vacuum on the enclosed system, and then the sample was added to the flask. Within 2 hours of adding the sample, all nitric oxide was released from the sample and quantified. To accelerate nitric oxide release, 60 W irradiation was employed in combination with sonication and heat. The distance between light source and the sample flask was maintained at 10 cm or less. A heat gun was used as the heat source during measurements.

### Example 9. Milling of Particles

Particles were milled in a lab-scale planetary ball mill (Fritsch Pulvirisette Classic P6) that rotates in planetary fashion with speeds as high as 650 RPM. The ball mill consists of a 12 ml or 80 ml milling cup made of Zirconium Oxide (ZrO₂) and contains ZrO₂ milling beads of 1 mm diameter. (Milling beads may be as small as 0.1 mm diameter and as large as 10 mm diameter, but 1 mm beads were used here). In order to achieve extensive particle size reduction, a solvent was added to the milling chamber, which in this case was water; however, water or any alcohol may be suitable. Enough water was added to achieve oil-like consistency, which is ideal for maximum particle size reduction. For the 12 ml grinding cup, an ideal mixture would be 20 g of 1 mm beads, 1 g of silicate powder, and ˜4 ml water or ethanol. It is also ideal to mill this mixture at high speed for 5 minutes and inspect the mixture to determine if more water or ethanol needs to be added because of the increased surface area of the particles absorbing more of the grinding solvent. The milling procedure involved 4 cycles of milling, wherein each cycle consisted of 4 min milling at 650 rpm followed by 4 min pause to allow for cooling. When milling was complete, the milled particles were removed from the ZrO₂ milling cup and separated from the 1 mm milling beads.

### Example 10. NO Release Using Simulated Skin Conditions

NO release from compositions including particles loaded with NO was evaluated to simulate the spreading that would occur when applied topically. For this simulation, a composition including dry particles loaded with NO was incorporated into a petroleum vehicle. The composition included dry S-nitrosated particles mixed with an anhydrous petrolatum delivery vehicle, the particles present at 25% by weight of the composition. The composition was then immediately applied to a piece of wax paper having a surface area of 6 cm² and spread thinly to simulate topical application. The wax paper was then transferred to a sampling chamber held at 34° C. and NO release was monitored. The sampling chamber was isolated from outside lighting and placed under a high precision LED microscope white light lamp with adjustable setting (AmScope LED-6WD); lux was measured using a digital lux meter (Dr. Meter, Model: LX1010B). Levels of nitric oxide were measured every minute for a period of 60 hours.

### Example 11. NO Release in Aqueous Conditions

NO release in aqueous conditions was evaluated to simulate alternative delivery routes such as intravenous administration. For this, a 50 mg aliquot of S-nitrosated particles was added to 5 mL of phosphate buffer in a sampling chamber at 37° C. which was connected to a nitric oxide analyzer. Levels of nitric oxide were measured every 30 seconds for a period of 12 hours.

### Results

Results provided in the drawings and described herein are meant to be exemplary and are not intended to limit the methods and compositions to modifications or alternatives as would be understood by a person of ordinary skill in the field of endeavor.

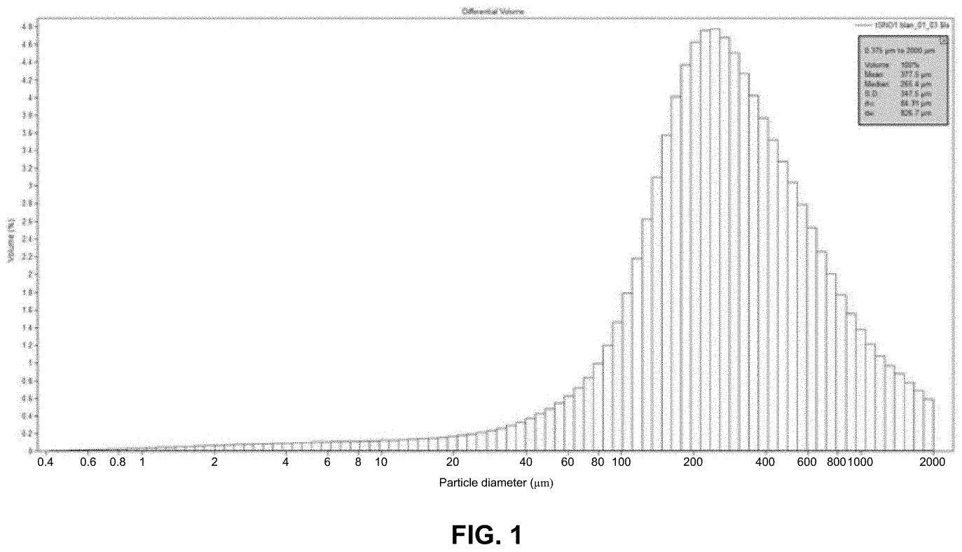

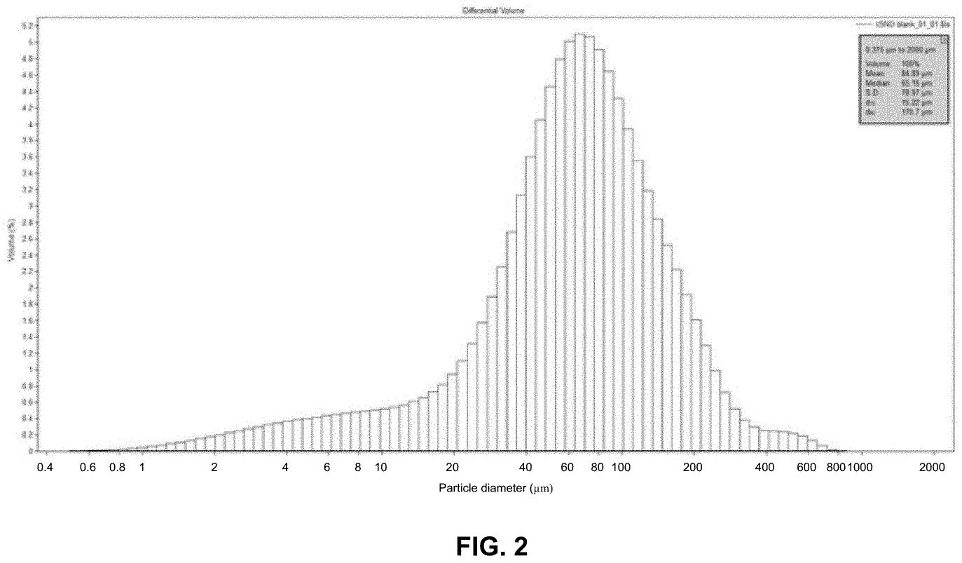

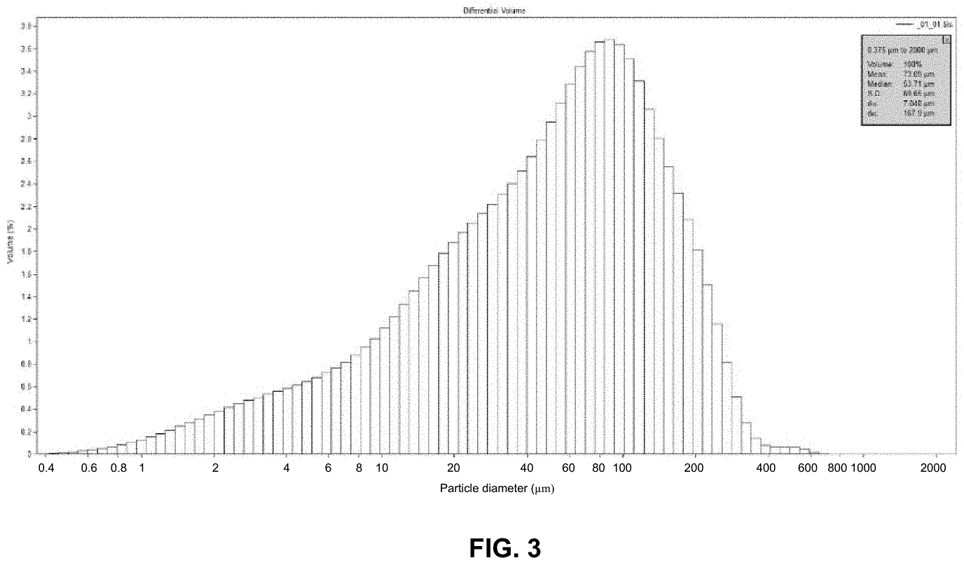

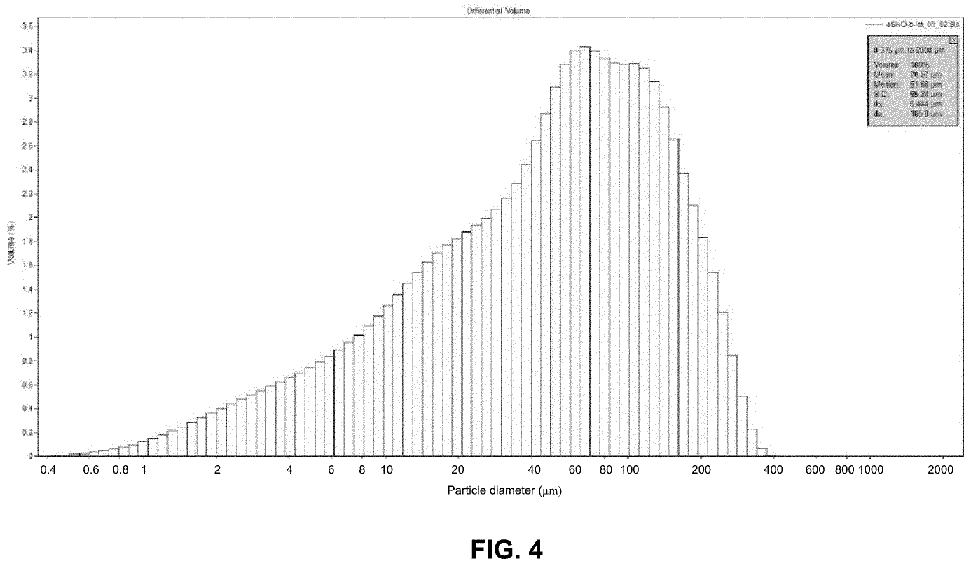

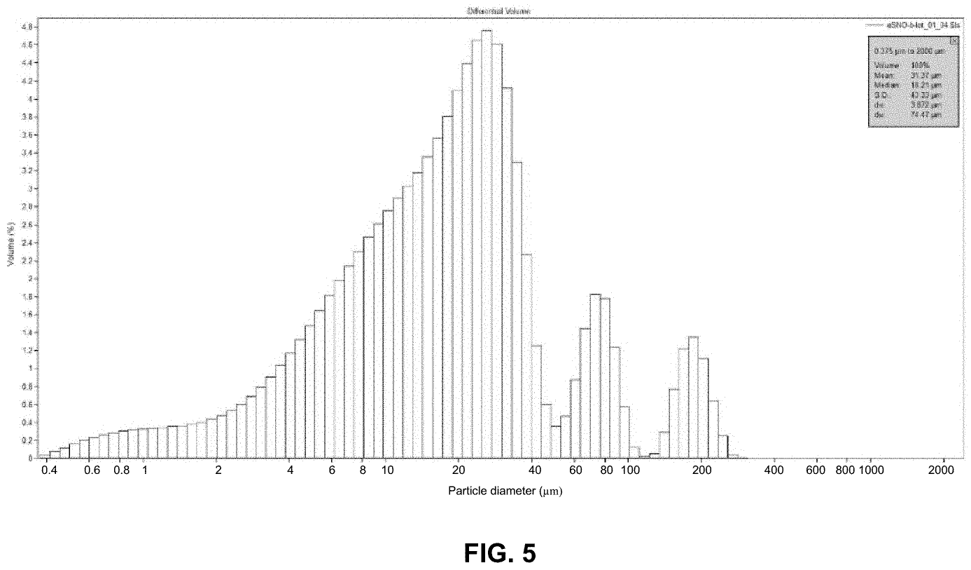

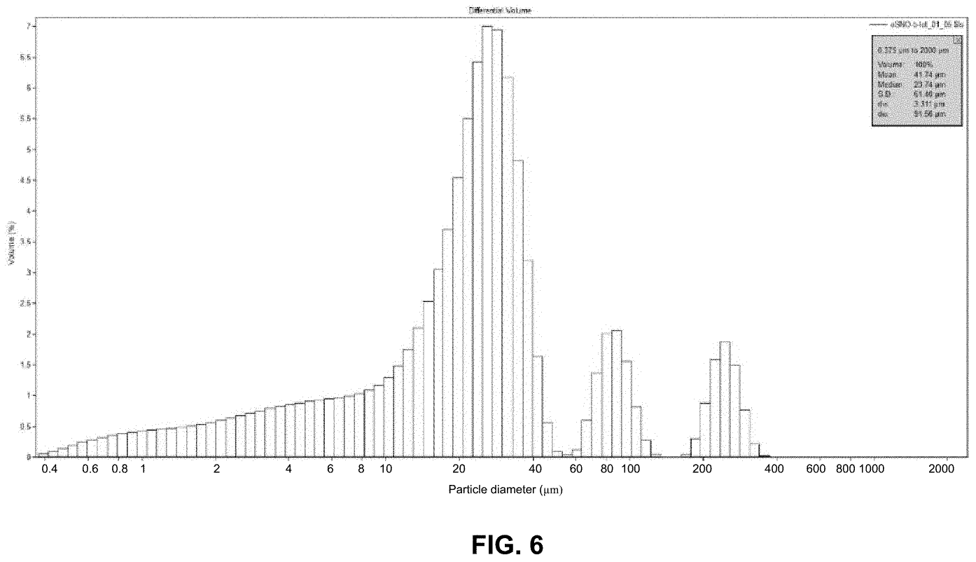

Referring now to FIGS. 1-6 each of these figures illustrates a graph displaying a histogram of the particle size distribution for thiol-functionalized (blank) silica particles prepared from co-condensation of hydrolyzed TEOS and MPTS.

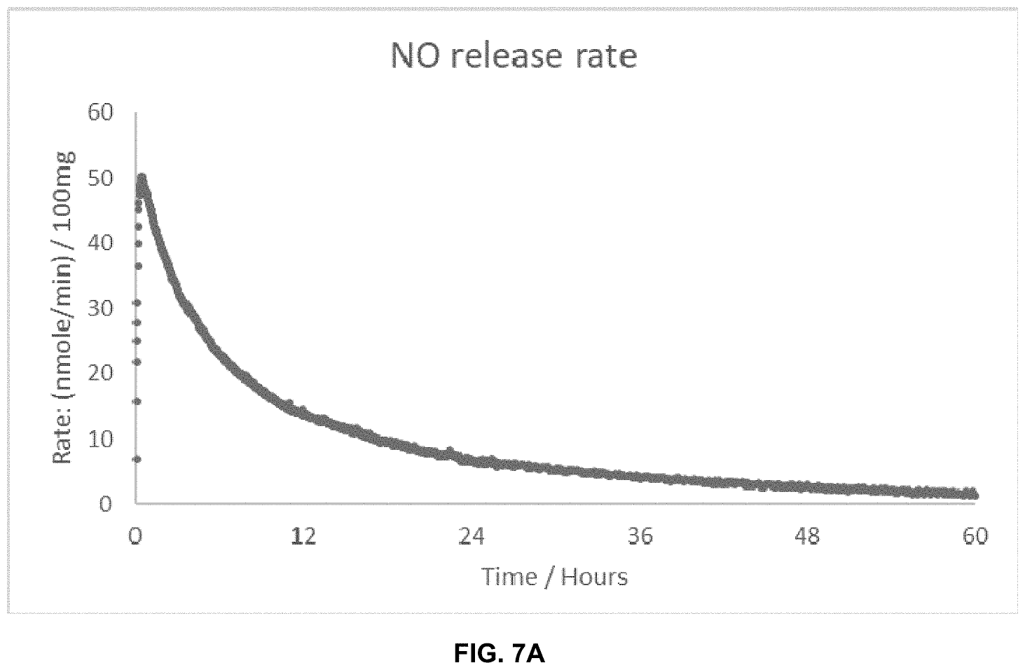

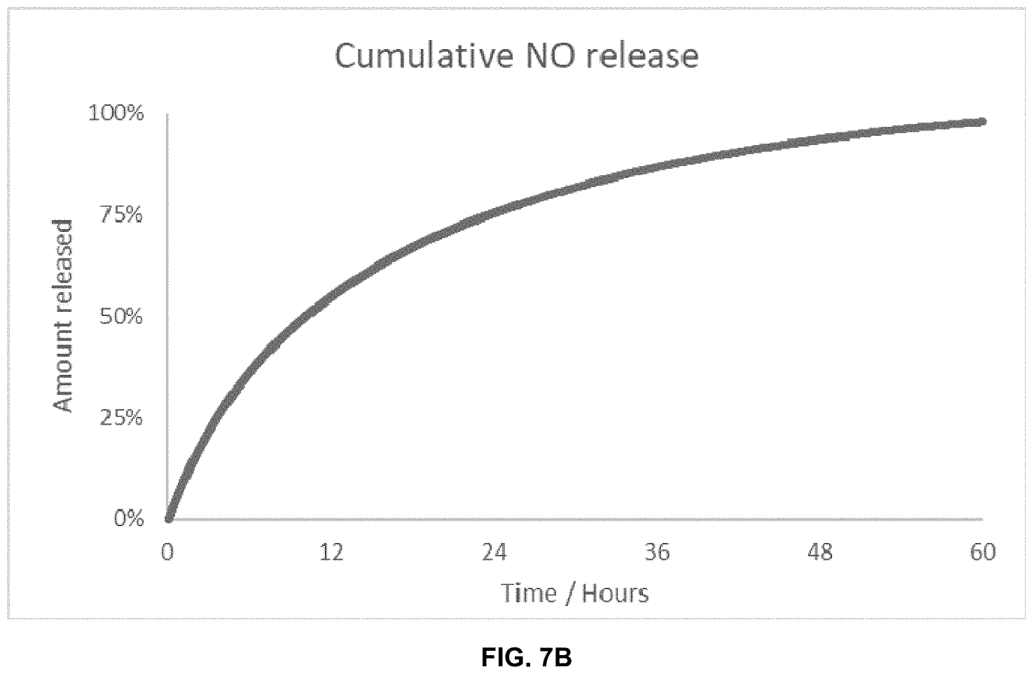

Referring now to FIGS. 7A and 7B, these images illustrate graphs displaying the NO release rate and cumulative release as measured using the conditions described in NO release using simulated skin conditions section of the examples. The graphs illustrate that at physiologically relevant conditions, controlled NO release can be achieved over an extended period of time, with the majority (50% or more) of NO being released within the first 12 hours.

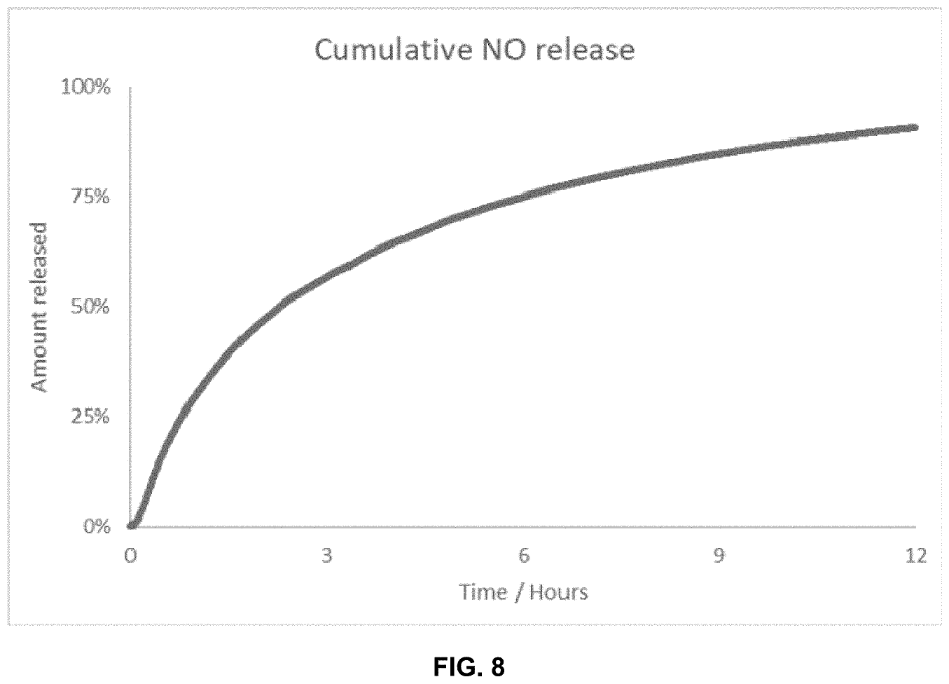

Referring now to FIG. 8, the image illustrates a graph displaying the cumulative NO release as measuring using the conditions described in the NO release in aqueous conditions section of the examples. The graph illustrates that in solution, NO releases faster compared to topical administration with the majority (50% or more) of NO being released within the first 3 hours.

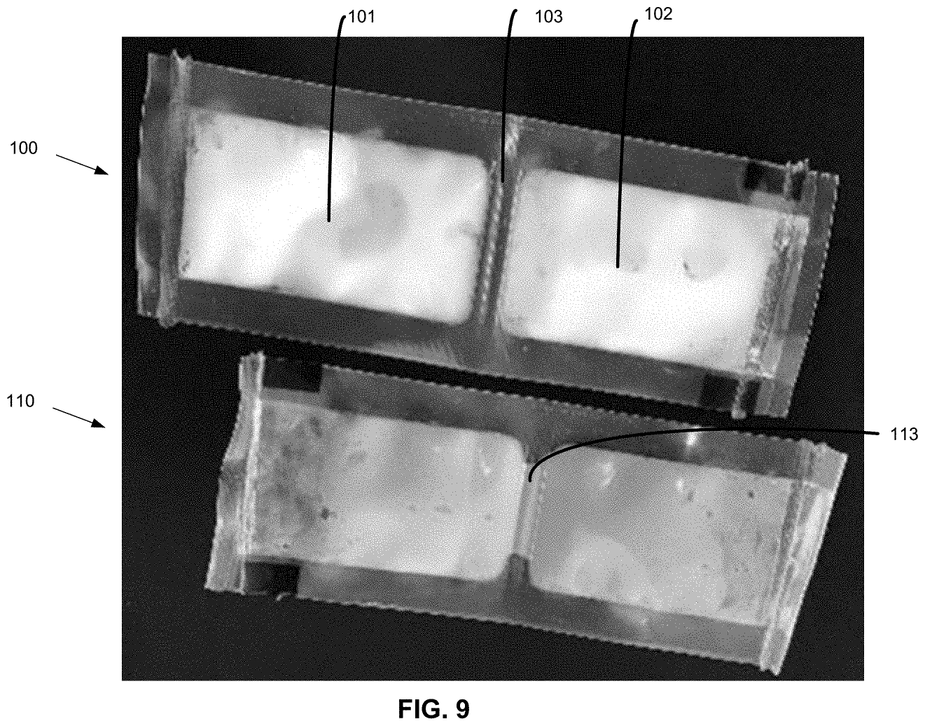

Referring now to FIG. 9, the photograph displays an example embodiment of a of a multi-compartment delivery system before 100 disruption of the barrier 103 and after 110 disruption of the barrier and subsequent mixing of the two compositions. The image displays two compartments: a first compartment 101 containing a first composition and a second compartment 102 containing a second composition separated by a frangible barrier 103. Upon disruption of the frangible barrier 103, material contained in the first compartment 101 can mix with material contained in the second compartment 102 to generate NO as indicated by the color change from white to red or pink (shown here in grayscale as gray.)

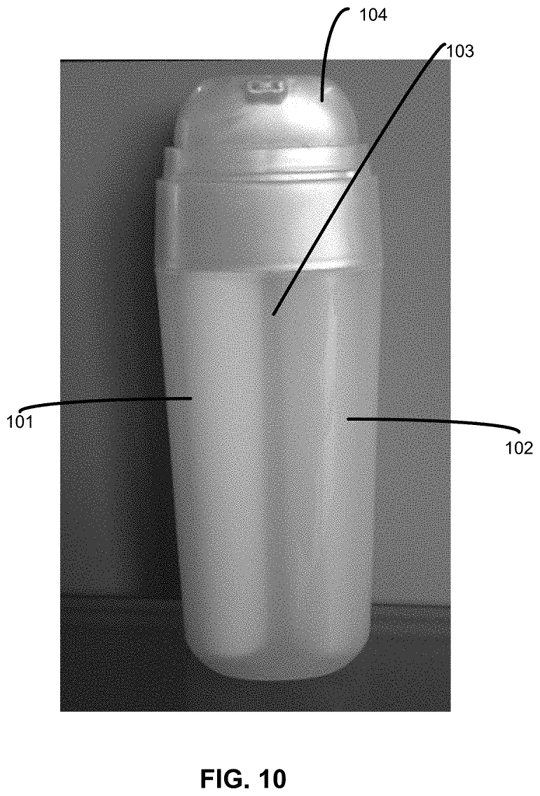

Referring now to FIG. 10, the photograph displays another example embodiment of a multi-compartment delivery system. The image displays a pumping system having two compartments: a first compartment 101 containing a first composition and a second compartment 102 containing a second composition separated by a barrier 103. Upon applying pressure to the delivery system which includes a dispenser 104, material from the first compartment 101 and material from the second compartment 102 are expelled from the dispenser and can subsequently be mixed to generate NO by an end user.

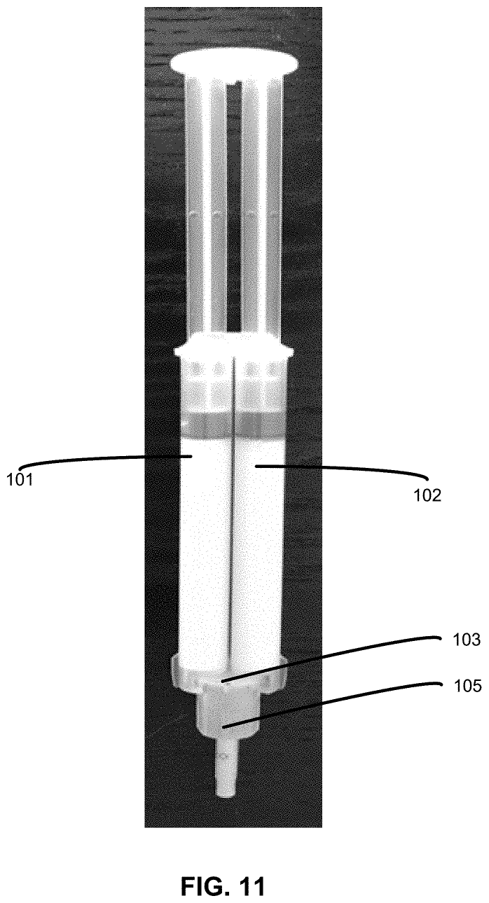

Referring now to FIG. 11, the photograph displays another example embodiment of a multi-compartment delivery system. The image displays a two-barrel syringe having two compartments: a first compartment 101 containing a first composition and a second compartment 102 containing a second composition separated by a barrier 103. Upon applying pressure to the delivery system which includes a mixing region 105, material from the first compartment 101 contacts material from the second compartment 102 to generate NO, and can be subsequently expelled from the dispenser to an end user.

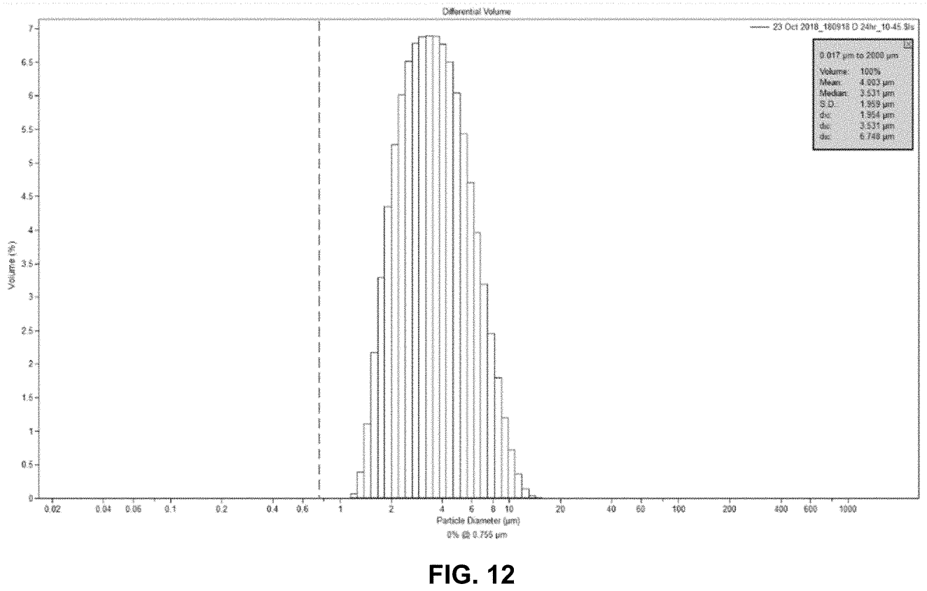

Referring now to FIG. 12, this figure illustrates a graph displaying a histogram of the particle size distribution for thiol-functionalized (blank) silica particles prepared from co-condensation of hydrolyzed TEOS and MPTS as described in example 5.

1. A method for generating NO by admixing at least two compositions, the method comprising:
obtaining a first composition comprising a thiol-bearing species;
obtaining a second composition comprising one or more compounds, and
mixing a portion of each composition together,
wherein one of the at least two compositions comprises a nitrite.

2. The method of claim 1, wherein one or more of the at least two compositions further include a carrier.

3. The method of claim 2, wherein the carrier comprises petrolatum and/or aloe Vera.

4. The method of claim 1, wherein the thiol-bearing species comprises a plurality of particles, and wherein each particle includes one or more thiol groups on the particle surface.

5. The method of claim 4, wherein the plurality of particles is polydisperse.

6. The method of claim 1, wherein the thiol-bearing species comprises a polymer or a compound.

7. The method of claim 1, wherein the first composition further comprises the nitrite and an acid, and wherein the first composition is substantially anhydrous.

8. The method of claim 8, wherein the one or more compounds comprise water.

9. The method of claim 1, wherein the one or more compounds comprise an acid.

10. The method of claim 1, wherein the first composition further comprises an acid.

11. The method of claim 10, wherein the one or more compounds comprise the nitrite.

12. A multi-chamber delivery system comprising:
a first chamber containing a polydisperse mixture of thiol-bearing particles,
a second chamber containing one or more compounds, and
a barrier, wherein the barrier is configured to physically prevent material contained in the first chamber from contacting material contained in the second chamber unless a condition is satisfied.

13. The multi-chamber delivery system of claim 12, wherein the barrier comprises a frangible seal, and wherein the condition includes a shear force.

14. The multi-chamber delivery system of claim 13, wherein the one or more compounds comprise a nitrite.

15. A method for extended release delivery of nitric oxide to a biological surface, the method comprising:
obtaining a first composition containing a thiol-bearing species;
obtaining a second composition containing one or more compounds;
exposing a portion of the first composition to an amount of the second composition to generate a mixture;
applying the mixture, the first composition, or the second composition to the biological surface.

16. The method of claim 15, wherein the biological surface includes one or more of the following: a skin surface, a mucous membrane surface, a keratin surface.

17. The method of claim 15, wherein the one or more compounds comprise a nitrite.

18. The method of claim 15, wherein the thiol-bearing species comprises a plurality of particles and wherein each particle includes one or more thiol groups on the particle surface.

19. The method of claim 18, wherein the plurality of particles is polydisperse.

20. The method of claim 15, wherein applying the mixture, the first composition, or the second composition to the biological surface further comprises treating a condition included in the group: inflammation, cardiac ischemia, erectile dysfunction, atopic dermatitis, and cancer.
